(19) United States
(12) Patent Application Publication
(10) Pub. No.: US 2024/0353586 A1
(43) Pub. Date: Oct. 24, 2024

(54) GROUND SENSOR

(71) Applicant: Geoprospectors GmbH, Traiskirchen (AT)

(72) Inventor: Michael Pregesbauer, Baden (AT)

(21) Appl. No.: 18/684,559

(22) PCT Filed: Aug. 17, 2022
(86) PCT No.: PCT/AT2022/060286

§ 371 Date: Feb. 16, 2024

(30) Foreign Application Priority Data
Aug. 20, 2021 (AT) A50678/2021

(51) Int. Cl. G01V 3/10; A01B 76/00; A01B 79/00; G01V 3/165

(52) U.S. Cl. CPC G01V 3/107; A01B 76/00; G01N 33/245; G01V 3/165; A01B 79/005

(57) ABSTRACT

The invention relates to a ground sensor (1) for detecting the quality of a substratum, comprising at least one electromagnetic transmission coil (2) and at least one pair of electromagnetic receiving coils (3, 3′), wherein the transmission coil (2) is designed to generate an electromagnetic primary field, and the receiving coils (3, 3′) are designed to receive an electromagnetic secondary field induced in the substratum by the primary field. The ground sensor (1) has a central plane E, the transmission coil (2) is arranged in the region of the central plane E, and the receiving coils (3, 3′) are arranged at a distance d₁ which is equidistant to the central plane E.

The invention relates to a ground sensor for detecting the quality of a substratum.

WO 2017/092885 A1 discloses a ground sensor for detecting the quality of a substratum. The sensor is based on a geophysical measurement system, the principle of electromagnetic induction (EMI), and is used to detect the quality of a substratum. A transmission coil generates a primary field, which induces a secondary field depending on the condition of the substratum. The close surface area of a few centimetres to several metres can be detected, depending on the configuration of the coil. Since EMI technology, unlike other geophysical methods, can be used in almost all ground situations and is also comparatively inexpensive to purchase, it has established itself as a measurement method in site-specific agriculture (so-called precision farming).

However, mounting such a highly sensitive EMI measurement system on a vehicle results in undesirable interactions with the vehicle. Specifically, the vehicle causes an electromagnetic interference field that interacts with the primary field generated by the measurement system. An interference flow is generated that must be dealt with separately in the data processing. The desired signal, which is relatively weak in relation to the interference signal, can become completely unusable by unfavourable mounting of generic ground sensors on the vehicle.

A further problem arises when measuring on highly resistive types of ground. Here, the low conductivity of the ground also leads to an unfavourable signal-to-noise ratio. In combination with the high ratio of noise caused by the interference field of the working machine, measurement data can become unusable in the worst case.

The object of the invention is to solve these and other problems.

These and other tasks are solved by the ground sensor according to claim 1.

A ground sensor for detecting the quality of a substratum according to the invention comprises at least one electromagnetic transmission coil and at least one pair of electromagnetic receiving coils. The transmission coil is configured to generate an electromagnetic primary field and the receiving coils are configured to receive an electromagnetic secondary field induced in the substratum by the primary field. The ground sensor has a central plane E. In particular, the central plane E may be a plane with the same distance to two lateral dimensions of the ground sensor; it may also be a symmetrical plane with regard to the outer shape of the ground sensor.

Preferably, the ground sensor may be attached to an implement in the area of this central plane. According to the invention the transmission coil is arranged in the region of the central plane E and the receiving coils are arranged on both sides at a substantially equal distance d₁ to the central plane E.

This results in the advantages according to the invention that the induced electromagnetic field can be measured equidistant to the transmission coil at the same time. This results in a doubling of the measurement signal. If the ground conditions have small-scale local differences between the positions of the receiving coils, the two received signals can also be averaged.

It may be provided that two, three or more pairs of electromagnetic receiving coils are provided, each of which is arranged equidistantly, i.e. at equal distances, to the central plane E.

This results in the advantages according to the invention that a selection can be made during the measurement as to which measurement signal is to be taken into account. Receiving coils that are closer to the central plane E have a higher interference signal when used as intended, as they are closer to the motor of the working machine. At the same time the receiving coils have a lower exploration depth, as they are close to the transmission coil. On the contrary, receiving coils with greater distance to the central plane E have a lower interference signal and a greater exploration depth. The arrangement of several equidistantly arranged pairs of receiving coils according to the invention allows certain pairs of receiving coils to be selected depending on the desired exploration depth and acceptable signal-to-noise ratio (SNR).

According to the invention, it may be provided that the distances between the adjacent receiving coils are equal to the distance between the transmission coil and the closest receiving coil.

This results in the advantage according to the invention that a symmetrical area around the transmission coil is evenly covered by the receiving coils.

In particular, it may be provided according to the invention that the receiving coils are rigidly arranged in relation to the transmission coil. Alternatively, it may be provided, however, that the receiving coils are moveably arranged in relation to the transmission coil, for example, on moveable carriages, which may be fixed to a fixed frame or the like using latches. This may vary the spatial resolution or exploration depth of the measurement on site. For example, pre-calibrated latches may be provided equidistant to the central plane with an accuracy of less than 0.5 mm, wherein the movable carriages ensure torsion-free movement of the receiving coils to the latches.

According to the invention, it may be provided that d₁ is equal to 20 cm to 80 cm, preferably 40 cm. In practice, it has been shown that this clearance provides particularly advantageous results and is suitable for suppressing some of the undesirable interactions with the working machine.

According to the invention, it may be provided that the transmission coil is configured to generate a magnetic field with a frequency in the range of 5 kHz to 30 KHz, in particular 8 kHz to 16 KHz.

According to the invention, it may be provided that the transmission coil and the receiving coils are arranged horizontally coplanar.

This results in the advantages according to the invention that, when used as intended, the main magnetic field generated by the transmission coil penetrates the ground and the induced measurement field can be measured particularly favourably by the receiving coils. The dimensionless induction number β can be used to calculate the penetration depth δ of the primary field into the ground. This is expressed by the equation

$$β = \sqrt{\frac{ω {μ}_{0} σ {s}^{2}}{2}} ≪ 1 ,$$

where ω is the frequency, μ₀ the magnetic field constant, σ the specific electrical conductivity and s the distance between the transmission coil and the receiving coil. For small values, the induction number β is approximately the ratio of the coil distance s to the penetration depth δ, so that different penetration depths δ can be selected by choosing different coil distances s. Thus, the penetration depth can be deduced from the distance of the receiving coils.

According to the invention, it may be provided that the transmission coil and the receiving coils each have turns with a wire diameter of approximately 0.7 mm, wherein the ratio of the number of turns of the transmission coil to the number of turns of the receiving coils is approximately 100:700. Here too, practice has shown that these values deliver particularly advantageous results.

According to the invention, it may be provided that the induced measurement voltage is in the range of approximately 9 nV at a frequency of 4 kHz and the magnetic flux density is in the range of 10⁻¹³ T.

According to the invention, it may be provided that the transmission coil and the receiving coils have a diameter ranging from 10 cm to 50 cm, particularly 25 cm to 40 cm. In practice, these dimensions are particularly advantageous for attaching the ground sensor to commercially available working machines.

Further, it may be provided according to the invention that a lock-in amplifier is provided to amplify the magnetic field signal received by the receiving coils.

According to the invention, two, three or more pairs of electromagnetic receiving coils and a multiplexer, which is configured to interrogate the receiving coils in pairs in chronological order, may be provided. The interrogation of the pairs of receiving coils can preferably take place at a frequency in the range of 10 Hz.

This has the advantage that measurement results from pairs of coils at different distances to the transmission coil are available, each of which can be used separately to compensate for vehicle interference. A switching frequency of the multiplexer in the range of 10 Hz achieves a sampling rate that makes sense in practice for slow-moving working machines.

According to the invention, a data processing unit may be provided which is configured to calculate the signal-to-noise ratio (SNR) of the signals received by the receiving coils and to select the pair of receiving coils which has the highest signal-to-noise ratio. This has the advantage that the measurement results can be filtered according to their SNR, and the measurement results with the lowest SNR are preferably used.

According to the invention, an agricultural working machine, for example a tractor, may be provided that comprises a ground sensor according to the invention. Such a working machine allows for precise analysis of the substratum during use. The data from the analysis can be used to operate and optimise site-specific agriculture.

According to the invention, the ground sensor may be attached to a linkage at the front or the rear of the working machine. In particular, the ground sensor may be attached to the working machine centred in relation to a longitudinal axis of the working machine. In particular, the ground sensor may be attached to the working machine in such a way that its central plane E is substantially central relative to the longitudinal axis of the working machine, so that the interference signals from a motor or drive, which is generally arranged centrally, act in the same way on the receiving coils arranged equidistantly to the central plane E.

Further features of the invention become apparent from the drawings, the specification of the embodiments and the figures. In the following, the invention is described in more detail with reference to the embodiments. In the figures:

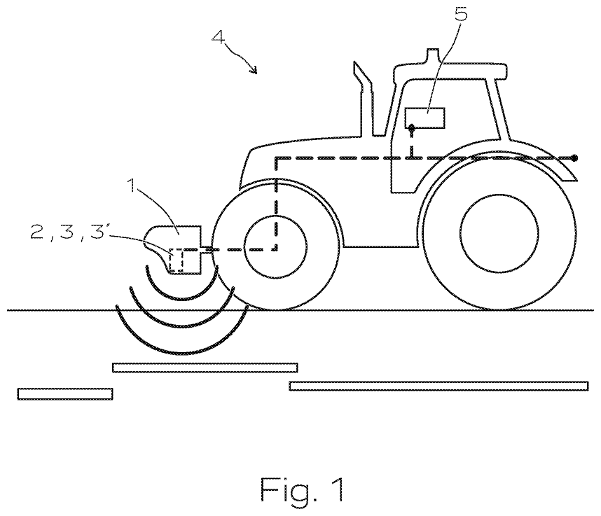

FIG. 1 is a schematic view of a working machine with an embodiment of a ground sensor according to the invention mounted on its front linkage.

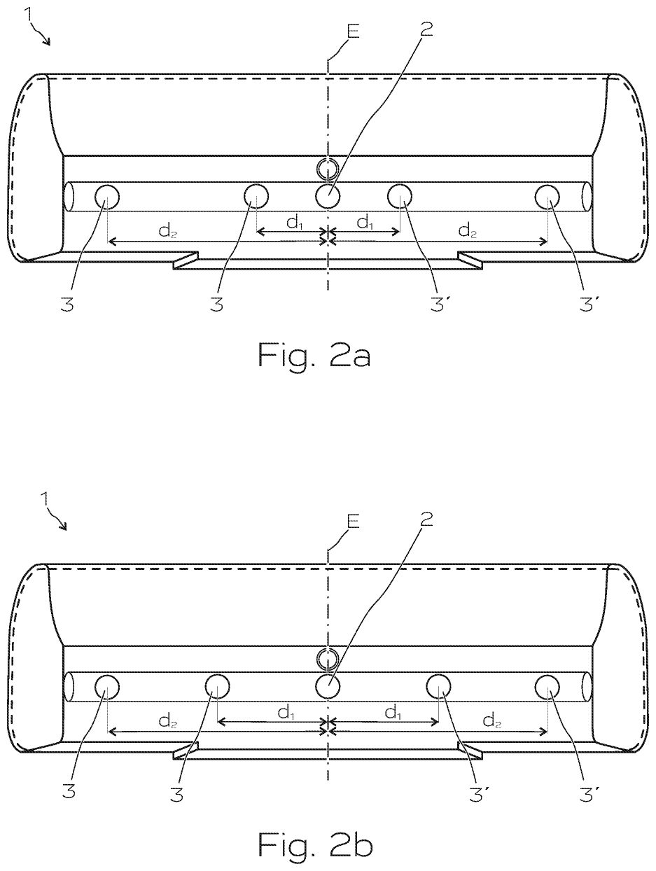

FIGS. 2a-2b show two different embodiments of a ground sensor according to the invention viewed from below;

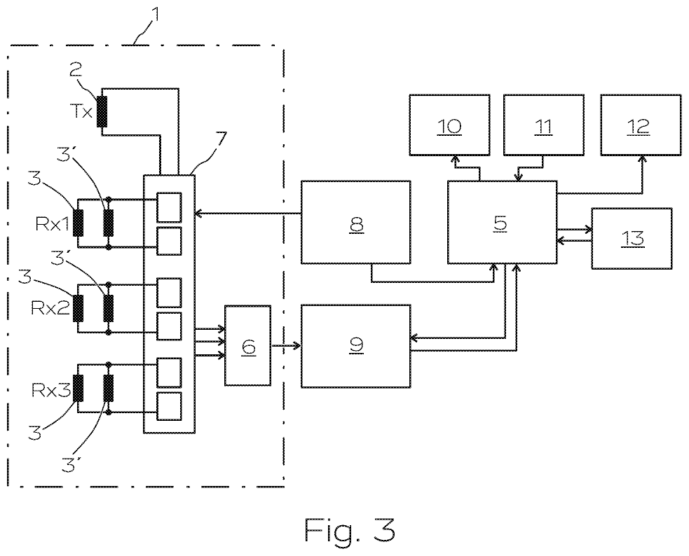

FIG. 3 shows a schematic circuit diagram of an electronic circuit for controlling a ground sensor according to the invention.

FIG. 1 shows a schematic view of a working machine 4, in this embodiment illustrated as a tractor, with a ground sensor 1 mounted on its front linkage. Schematically, a changing quality of the ground is shown in the direction of travel of the working machine 4. The electromagnetic signals transmitted by the ground sensor 1 through the transmission coil 2 are received by the ground and generate an induction field, which is received by the receiving coils 3, 3′.

For detecting the quality of the ground, the measured signals are transmitted to a data processing unit 5 located in the working machine 4 and processed therein. In this process, the working machine 4 is in motion so that an area of the substratum can be continuously analysed.

FIGS. 2a-2b show two different embodiments of a ground sensor 1 according to the invention viewed from below. The ground sensor 1 has a substantially cuboid shape and includes a coupling device for its attachment to an agricultural working machine. The ground sensor 1 comprises a central plane E. When the ground sensor 1 is used as intended, it is attached to a working machine and moved along the extent of the central plane E at a short distance over a ground.

The ground sensor 1 comprises a transmission coil 2 and two pairs of electromagnetic receiving coils 3, 3′. The transmission coil 2 and the receiving coils 3, 3′ are each configured as horizontal coplanar coils with a diameter of around 15 cm. In the present figure, the transmission coil 2 and the receiving coils 3, 3′ are normal to the plane of the drawing. The transmission coil 2 is configured to generate an electromagnetic primary field and the receiving coils 3, 3′ are configured to receive an electromagnetic secondary field induced by the primary field in the substratum.

The transmission coil 2 is arranged in the area of the central plane E. A first pair of receiving coils comprises two receiving coils 3, 3′ that are arranged on both sides at a substantially equal distance d₁ from the central plane E. The distance d₁ is approximately 40 cm. A second pair of receiving coils comprises two receiving coils 3, 3′ that are arranged on both sides at a substantially equal distance d₁ from the central plane E. In this embodiment d₂>2d₁, i.e. approximately 100 cm. In other words, in the embodiment of FIG. 2a the receiving coils 3, 3′ are each arranged equidistant to the transmission coil 2, however, the distance between adjacent receiving coils 3, 3′ is optionally different from the distance between the transmission coil 2 and the closest receiving coil 3, 3′.

The embodiment of FIG. 2b corresponds to that in FIG. 2a, although in this embodiment d₂=2d₁, i.e. approximately 80 cm. In other words, in embodiment of FIG. 2b the distances between adjacent receiving coils 3, 3′ are always equal to the distance between the transmission coil 2 and the closest receiving coil 3, 3′. As a result, a symmetrical area around the transmission coil 2 can be evenly covered by the receiving coils 3, 3′.

In other embodiments not described herein only one pair of receiving coils 3, 3′ is provided or more than two pairs of receiving coils 3, 3′ are provided.

FIG. 3 shows a schematic circuit diagram of a ground sensor 1 according to the invention including an electronic circuit for controlling the ground sensor 1. The ground sensor 1 comprises a transmission coil 2 referred to as Tx, and three pairs of receiving coils 3, 3′ referred to as Rx1, Rx2 and Rx3. The transmission coil 2 and the receiving coils 3, 3′ are connected to a TX/RX coil array 7 so that they can be activated independently of each other.

A frequency generator 8 is provided, which generates a periodic electrical transmission signal with a frequency in the range of 8 kHz-9 kHz, which is fed into a data processing unit 5 and into the TX/RX coil array 7. The transmission coil 2 uses this to generate a primary field for transmission into the ground.

The receiving coils 3, 3′ are interrogated in a chronological order by the multiplexer 6 at a frequency of approximately 10 Hz. The signals received by the receiving coils 3, 3′ are amplified by a lock-in amplifier and then transmitted to an A/D converter 9. The A/D converter transmits the translated signal to the data processing unit 5, where the 1D inversion is calculated to determine the ground parameters from the conductivity values received from the three pairs of receiving coils Rx1, Rx2, Rx3. The data processing unit 5 calculates a signal-to-noise ratio from the transmission signal and the receiving signals of the receiving coils 3, 3′ and selects that pair of receiving coils 3, 3′ with the highest signal-to-noise ratio.

Further, the data is stored in a memory unit 10. GPS data from the working machine is received by a GPS receiver 11 in order to determine the position of the ground sensor 1. A bus interface 12 is able to tap the data and there may be an exchange with a serial interface 13.

The scope of protection of the invention is not limited to the described embodiments but comprises all objects of the following claims. In particular, the scope of protection is not limited to ground sensors for use in agriculture.

### LIST OF REFERENCE SIGNS

1 ground sensor
2 transmission coil
3, 3′ receiving coil
4 working machine
5 data processing unit
6 multiplexer
7 TX/RX coil array
8 frequency generator
9 A/D converter
10 memory unit
11 GPS receiver
12 bus interface
13 serial interface

1. A ground sensor (1) for detecting the quality of a substratum, comprising at least one electromagnetic transmission coil (2) and at least one pair of electromagnetic receiving coils (3, 3′), wherein the transmission coil (2) is configured to generate an electromagnetic primary field, and the receiving coils (3, 3′) are configured to receive an electromagnetic secondary field induced by the primary field in the substratum, wherein the ground sensor (1) has a central plane E,
characterised in that
the transmission coil (2) is arranged in the area of the central plane E, and
the receiving coils (3, 3′) are arranged on both sides at a substantially equal distance d₁ from the central plane E.

2. The ground sensor (1) according to claim 1, characterised in that two, three or more pairs of electromagnetic receiving coils (3, 3′) are provided, each of which is arranged equidistantly to the central plane E.

3. The ground sensor (1) according to claim 2, characterised in that the distances between adjacent receiving coils (3, 3′) are equal to the distance between the transmission coil (2) and the closest receiving coil (3, 3′).

4. The ground sensor (1) according to claim 1, characterised in that the distance d₁ is equal to 20 cm to 80 cm, preferably 40 cm.

5. The ground sensor (1) according to claim 1, characterised in that the transmission coil (2) is configured to generate a magnetic field with a frequency in the range of 5 kHz to 30 kHz, preferably 8 kHz to 16 kHz.

6. The ground sensor (1) according to claim 1, characterised in that the transmission coil (2) and the receiving coils (3, 3′) are arranged horizontally coplanar.

7. The ground sensor (1) according to claim 1, characterised in that the transmission coil (2) and the receiving coils (3, 3′) have turns with a wire diameter of approximately 0.7 mm, wherein the ratio of the number of turns of the transmission coil (2) to the number of turns of the receiving coils (3, 3′) is approximately 100:700.

8. The ground sensor (1) according to claim 1, characterised in that the transmission coil (2) and the receiving coils (3, 3′) each have a diameter ranging from 10 cm to 50 cm, in particular from 25 cm to 40 cm.

9. The ground sensor (1) according to claim 1, characterised in that a lock-in amplifier is provided to amplify the magnetic field signal received by the receiving coils (3, 3′).

10. The ground sensor (1) according to claim 1, characterised in that two, three or more pairs of electromagnetic receiving coils (3, 3′) are provided and a multiplexer (6) is provided that is configured to interrogate the receiving coils (3, 3′) in pairs in a chronological order, preferably with a frequency in the range of 10 Hz.

11. The ground sensor (1) according to claim 9, characterised in that a data processing unit (5) is provided which is configured to calculate the signal-to-noise ratio of the signals received by the receiving coils (3, 3′) and to select that pair of receiving coils (3, 3′) which has the highest signal-to-noise ratio.

12. An agricultural working machine (4) comprising a ground sensor (1) according to claim 1.

13. The agricultural working machine (4) according to claim 12, characterised in that the ground sensor 1 is attached to a linkage at the front or rear of the working machine.

14. The agricultural working machine (4) according to claim 12, characterised in that the ground sensor (1) is attached to the working machine centred in relation to a longitudinal axis of the working machine, preferably in a way that the central plane E extends substantially centred relative to the longitudinal axis.
